(19) United States
(12) Patent Application Publication
(10) Pub. No.: US 2026/0210835 A1
(43) Pub. Date: Jul. 23, 2026

(54) INTEGRATED ANALYSIS SYSTEM AND METHOD FOR THE RAPID MEASURING AND RECORDING OF DATA FROM A HOST

(71) Applicant: Hugh Califf, Downers Grove, IL (US)

(72) Inventor: Hugh Califf, Downers Grove, IL (US)

(21) Appl. No.: 19/541,283

(22) Filed: Feb. 16, 2026

Related U.S. Application Data
(63) Continuation-in-part of application No. 18/530,167, filed on Dec. 5, 2023.

(51) Int. Cl. G01N 15/1434; A61B 5/15; G01N 15/1429; G01N 27/447; G01N 33/49; G01N 33/72

(52) U.S. Cl. CPC G01N 15/1434; A61B 5/150229; A61B 5/150755; A61B 5/150847; G01N 15/1429; G01N 27/447; G01N 33/4915; G01N 33/721

(57) ABSTRACT

An integrated analysis system for the rapid analysis, recording, and transmission of host biometric data is provided. The method comprises introducing an aqueous biological material through a vertical passageway into a fully enclosed flow chamber, where a central hub distributes the material radially through integrated flow channels. The material is received into microtubes and analyzed by embedded medical diagnostic sensors to simultaneously measure physical and chemical parameters, including quantitative optical measurements, without prior analyte concentration or separation. The method includes storing collected data on an internal memory cartridge and executing real-time predictive analytics via a remote AI processing unit in wireless communication with the system. Final diagnostic results are transmitted via a wireless transmitter, providing stationary sensor analysis from clinical specimen collection equipment, such as a manual user-actuated syringe, coupled to the chamber's injection point.

### RELATED APPLICATIONS

This application claims priority to U.S. Utility patent application Ser. No. 18/530,167 filed Dec. 5, 2023, now pending.

### FIELD OF THE INVENTION

The present disclosure relates to an integrated analysis system and method for the rapid measuring and recording of data from a host. More particularly, the subject matter relates to a fully enclosed flow chamber configured for the radial distribution of an aqueous biological material from a central distribution hub into a plurality of microtubes for stationary sensor analysis. The disclosure further relates to the integration of medical diagnostic sensors, memory cartridges, and remote access devices comprising artificial intelligence (AI) processing units configured for the wireless communication and predictive analytics of biometric data.

### BACKGROUND OF THE INVENTION

In the field of medical diagnostics, there is an increasing demand for accurate, rapid, and efficient systems capable of measuring and recording data from biological materials for tracking, measuring, and monitoring purposes. Conventional methods often involve time-consuming processes and may lack the ability to provide real-time and in-depth analysis of biological samples.

To address these challenges, various sensors and diagnostic tools have been developed to measure specific parameters of biological materials, such as density, flow rate, cellular features, and oxygen levels. Additionally, advancements in artificial intelligence have provided opportunities to enhance the analysis and interpretation of complex datasets derived from biological samples.

Furthermore, the integration of memory storage devices with analysis systems has become crucial for efficiently storing and accessing large volumes of data generated during the measurement and recording processes, enabling subsequent retrieval and analysis.

### SUMMARY

In order to accomplish the objectives of the present disclosure, there is provided a method for integrated blood analysis using a system that includes a fully enclosed flow chamber with a vertical passageway for introducing aqueous biological material via an injection point. The material is then distributed through a central hub, directed radially through flow channels, and received into microtubes containing embedded medical diagnostic sensors for analysis. Data from the sensors is stored on memory cartridges and processed wirelessly by a remote access device with AI units for predictive analytics and transmission of diagnostic results. The system uses a specific structural arrangement to prevent flow reversal and performs simultaneous physical and chemical parameter measurements. The method can use optical sensors for quantitative measurements without prior analyte concentration and supports tracking in vivo and in vitro parameters

Unless otherwise defined, all technical and scientific terms used herein have the same meaning as commonly understood by one of ordinary skill in the art. Although methods and materials similar or equivalent to those described herein can be used in the practice or testing of any described embodiment, suitable methods and materials are described below. In addition, the materials, methods, and examples are illustrative only and not intended to be limiting. In case of conflict with terms used in the art, the present specification, including definitions, will control.

The foregoing summary is illustrative only and is not intended to be in any way limiting. In addition to the illustrative aspects, embodiments, and features described above, further aspects, embodiments, and features will become apparent by reference to the drawings and the following detailed description and claims.

### BRIEF DESCRIPTION OF THE DRAWINGS

The present embodiments are illustrated by way of the figures of the accompanying drawings, which may not necessarily be to scale, in which like references indicate similar elements, and in which:

FIG. 1 is a hematologic analysis example of a complete blood count;

FIG. 2 is a secondary cellular analysis to detect the presence of a certain type of immune cell;

FIG. 3 shows the arrangement of the flow chamber and at least one microtube;

FIG. 4 shows the physical electronic connections between the flow chamber, sensors, memory cartridge, and an external monitoring device;

FIG. 5 shows the interplay of sensors used by AI, external monitoring devices, and physical electronic connections between the flow chamber, flow chamber sensors, memory cartridge, and an external monitoring device;

FIG. 6 shows an exemplary AI learning possibility of the positive or negative influences that inflammation can have on the microbiome and the impact of these influences on the brain;

FIG. 7 shows an exemplary biological material collection method using the flow chamber and the proximity of an external monitoring device;

FIG. 8 shows an exemplary biological material collection method using the flow chamber and the proximity of an external monitoring device;

FIG. 9 shows an exemplary biological material collection method using a clinical specimen collection device to load the flow chamber;

FIG. 10 shows an exemplary top view of the enclosed flow chamber; and

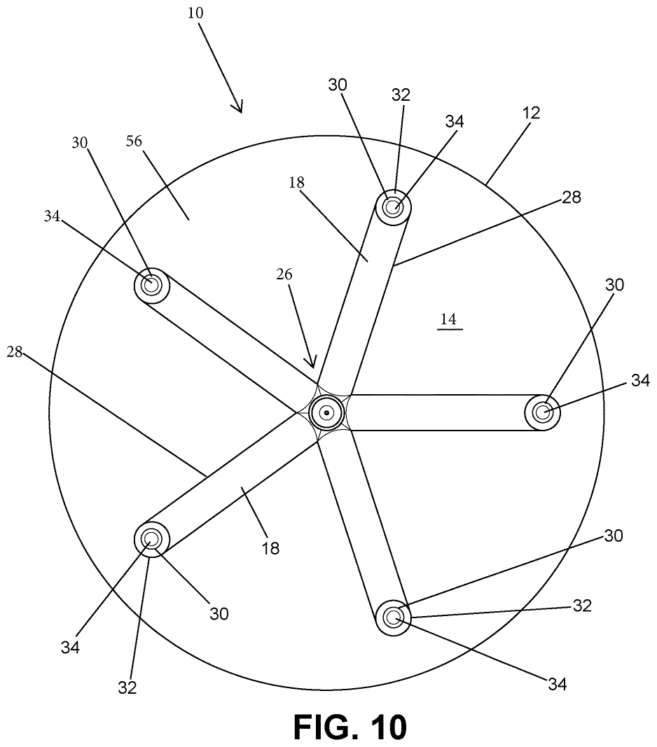

FIG. 11 shows an exemplary shows an exemplary bottom view of the enclosed flow chamber.

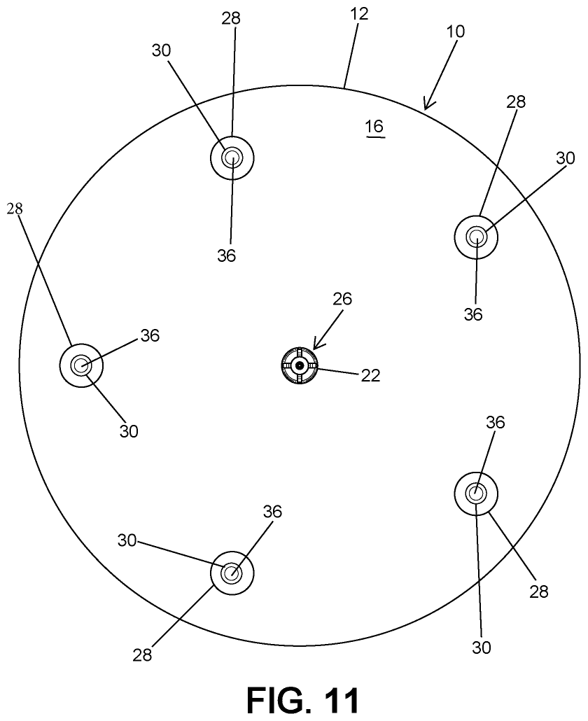

### DETAILED DESCRIPTION

The following detailed description is the best-contemplated mode of carrying out the disclosure. Although the disclosure has been explained in relation to its preferred embodiment, it is to be understood that many other possible modifications and variations can be made without departing from the spirit and scope of the invention.

### DEFINITIONS

To facilitate a clearer understanding of the integrated analysis system, the following definitions are provided for terms used throughout the specification and claims:

Aqueous biological material refers to any fluid specimen derived from a biological source, including but not limited to whole blood, plasma, serum, urine, saliva, or clinical samples in an aqueous carrier.

Central distribution hub refers to a structural distribution junction or hub within the flow chamber that receives fluid from a single input passageway and branches it out into multiple distinct flow channels.

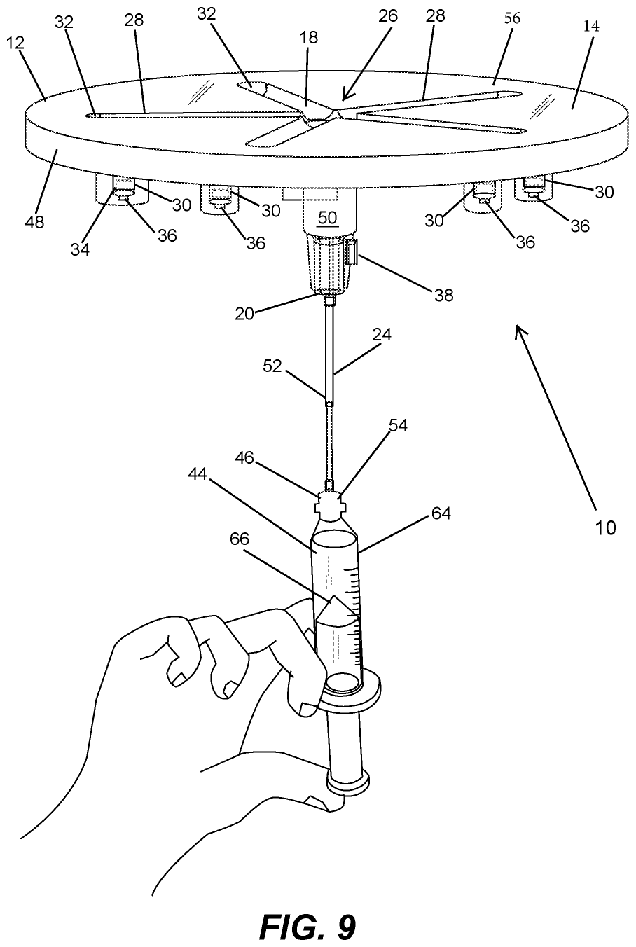

Pump force refers to the mechanical pressure applied to a fluid to initiate movement through the system, which may be generated by a manual plunger as shown in FIG. 9, element 66.

Stationary sensor analysis is a method of measurement where the biological material remains substantially at rest within a sensing compartment during the time period in which data is captured by the medical diagnostic sensors.

Artificial Intelligence (AI) Processing Units refers to hardware and software components, including machine learning algorithms and predictive analytics, configured to process complex biometric data to generate diagnostic results.

Hermetic seal is a vacuum-tight or air-tight structural connection that prevents the leakage of fluids and prevents the entry of external contaminants.

The present disclosure provides a method for integrated blood analysis using an integrated analysis system 10. The method begins by providing a fully enclosed flow chamber 12 having a top surface 14, a bottom surface 16, and an interior space 18 defined between the top 14 and bottom surfaces 16. An aqueous biological material 44 is introduced through a passageway 20 extending along a substantially vertical axis through the flow chamber 12, where the passageway 20 comprises a substantially circular hollow tube 22 defining an injection point 24 at the bottom surface 16. The method includes distributing the aqueous biological material 44 through a central distribution hub 26 located within the interior space 18 and fluidly connected to the passageway 20, and subsequently directing the material through a plurality of flow channels 28 monolithically integrated within the flow chamber 12 extending radially from the central distribution hub 26.

The method further comprises receiving the aqueous biological material 44 into a plurality of microtubes 30 located at outer ends 32 of the flow channels 28, where the microtubes 30 comprise interior compartments 34 housing at least one medical diagnostic sensor 36. In operation, the injection point 24 receives the aqueous biological material 44 from a clinical specimen collection equipment 46 removably attached thereto, such that a pump force moves the material from the injection point 24, through the central distribution hub 26, and into the plurality of microtubes 30 for stationary sensor analysis. The method utilizes a flow chamber 12 preferably composed of a biocompatible material 48. By positioning the plurality of microtubes 30 below a vertical plane 50 of the central distribution hub 26 and beneath the top surface 14, the method effectively prevents flow reversal of the aqueous biological material 44.

To ensure a secure environment, the method includes forming a hermetic seal at the injection point 24 via a female Luer lock connector 52 configured to receive a corresponding male Luer connector 54 of the clinical specimen collection equipment 46. A glass slide 56 is fixed horizontally across the top surface 14 to seal the radial arrangement of the flow channels 28 and microtubes 30. The method involves analyzing the aqueous biological material 44 via the medical diagnostic sensor 36, which may perform quantitative measurements as an optical sensor without prior analyte concentration or separation, or measure at least one physical parameter 68 and chemical parameter 68 simultaneously using electrochemical principles. Data is transferred between a memory cartridge 38 and the medical diagnostic sensor 36 via a local internal bus 58 within the sealed flow chamber 12.

For advanced data handling, the method includes processing data via a remote access device 40 comprising artificial intelligence processing units 42 in wireless communication with the memory cartridge 38 and the medical diagnostic sensor 36. The artificial intelligence processing units 42 execute predictive analytics to analyze biometric parameters 68 in real time and transmit final diagnostic results via a wireless transmitter 60. The method allows for individual preliminary analysis of the aqueous biological material 44 across at least five distinct analysis points via the plurality of microtubes 30. Furthermore, the method may include connecting at least one medical diagnostic sensor 36 to a host 62 to track in vivo and in vitro physical parameters 68. The pump force may be provided by coupling a pre-filled syringe 64 to the injection point 24 and actuating a manual user-actuated plunger mechanism 66.

The method is designed to rapidly capture measured signals 70 and communicate them through various interfaces 72 to enable timely results in emergency or remote settings. The medical diagnostic sensors 36 are exposed to biological materials 44 such as blood, urine, or saliva to measure parameters 68 such as analytes, blood gases, and hematocrit levels. The remote access device 40 utilized in this method may include a cell phone or computer for capturing and communicating the measured signals 70 from the medical diagnostic sensors 36.

### Micro-Electro-Mechanical Systems

The methodology of the integrated analysis system 10 can incorporate the use of Micro-Electro-Mechanical Systems (MEMS) technology to fabricate microscopic mechanical and electromechanical elements directly into the interior space 18 of the fully enclosed flow chamber 12. In such an embodiment, MEMS-based components could function as a medical diagnostic sensor 36 to perform real-time hemodynamic monitoring, thereby measuring the precise pressure and flow rate of the aqueous biological material 44 as it moves from the central distribution hub 26 into the microtubes 30. This approach potentially allows for high-accuracy cell counting by detecting electrical resistance changes as individual cells pass through micro-apertures within the interior compartments 34. As an alternative to MEMS, the system could utilize Microfluidic Surface Acoustic Wave (SAW) sensors to manipulate and sense particles using sound waves, or Photonic Crystal sensors to detect biological binding events through light-trapping structures.

### Electrophoresis

The methodology can further provide for the possible incorporation of electrophoresis to separate and quantify specific proteins or hemoglobin variants within the microtubes 30. By optionally applying a controlled electric field across the flow channels 28, the system could exploit the charge-to-size ratio of molecules to move them at different rates, facilitating the identification of specific parameters 68. In the context of the radial flow design, this electrophoretic process could serve as a secondary sorting step to refine analytes after they reach the interior compartments 34, allowing for highly sensitive quantitative measurements without requiring prior off-chip concentration. Alternatives to electrophoresis include dielectrophoresis (DEP), which uses non-uniform electric fields to move uncharged particles, and magnetophoresis, which utilizes magnetic bead tagging to pull specific analytes toward the medical diagnostic sensor 36 using external magnetic gradients.

### Heparinization

Additionally, the methodology could employ heparinization to maintain the fluid integrity of the aqueous biological material 44 throughout the analysis process. By optionally coating the interior surfaces of the fully enclosed flow chamber 12, the passageway 20, and the microtubes 30 with heparin, the system provides a means to prevent microthrombi that might otherwise block narrow channels and invalidate measured signals 70. This interaction could also facilitate the separation of plasma from red blood cells, ensuring that clear plasma reaches the medical diagnostic sensor 36 for more accurate analysis. Functional alternatives to heparinization include PEGylation (Polyethylene Glycol coating) to create a “stealth” surface that prevents protein adhesion, or Fluorinated Lubricant-Infused Surfaces (SLIPS) to prevent the aqueous biological material 44 from adhering to the biocompatible material 48 of the chamber walls entirely.

### Quantitative Measurements

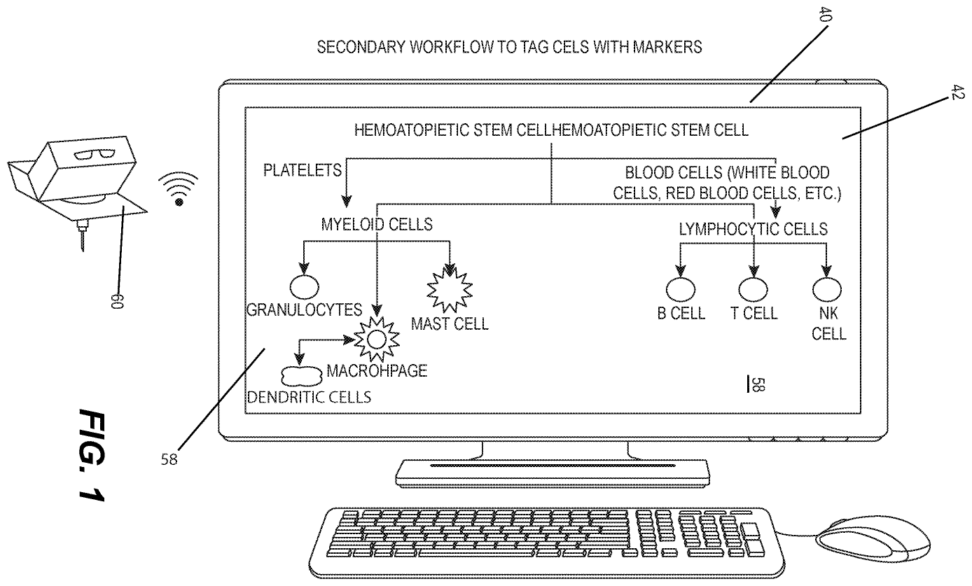

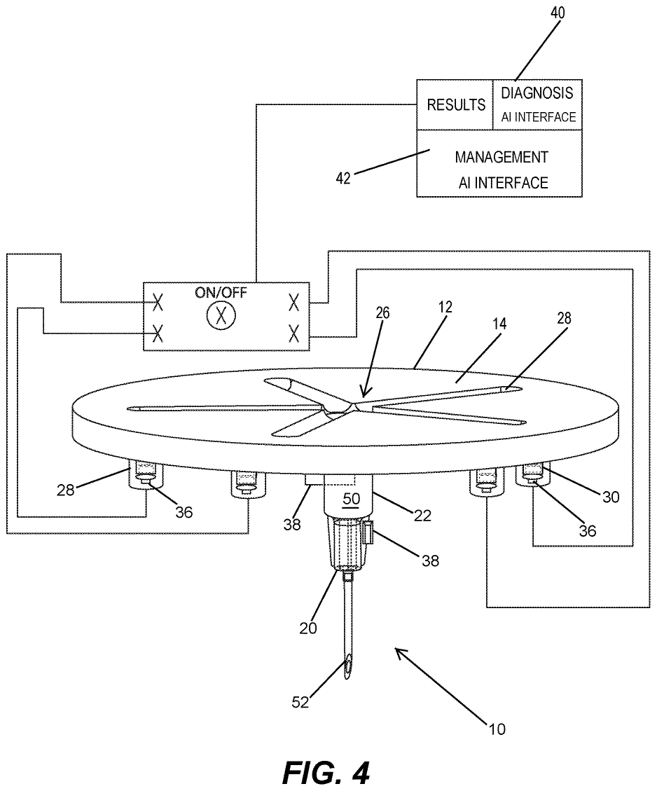

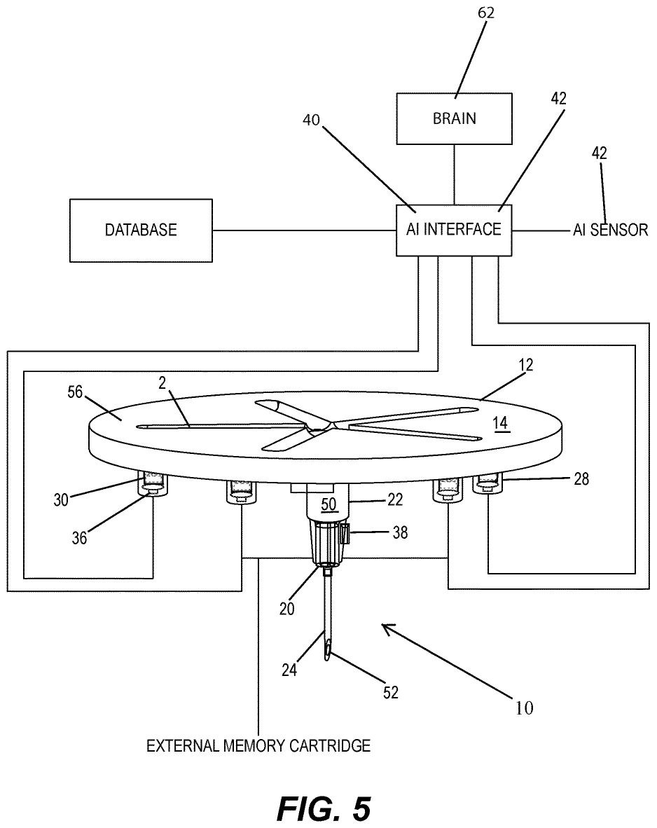

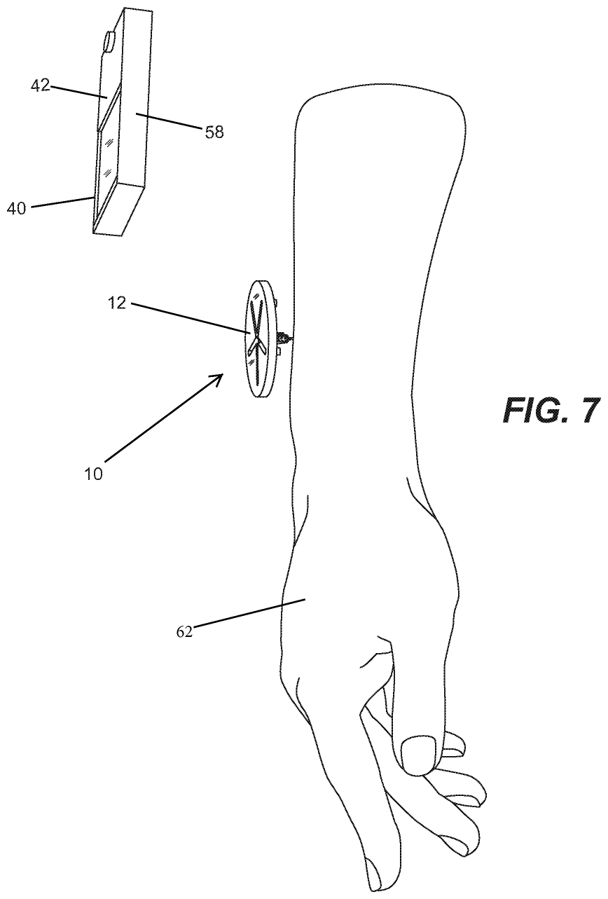

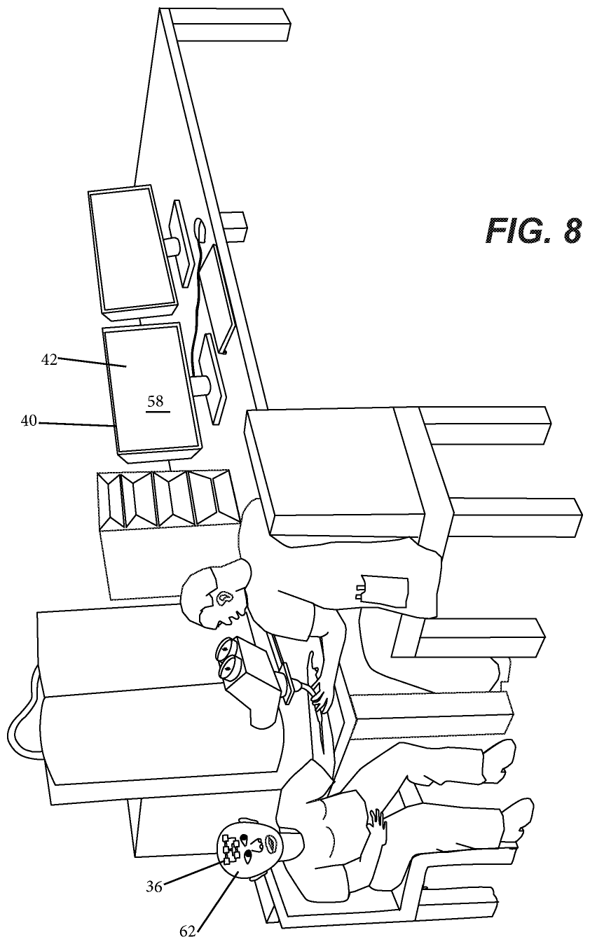

In a preferred implementation of the method, and as shown in FIGS. 1-9, the integrated analysis system 10 executes the rapid measuring and recording of data from a host 62. The method comprises utilizing a fully enclosed flow chamber 12 with a top surface 14 and an interior space 18 to perform quantitative measurements of aqueous biological material 44. The material is inserted into the flow channels 28 of the fully enclosed flow chamber 12 using clinical specimen collection equipment 46. The process involves housing medical diagnostic sensors 36 within the fully enclosed flow chamber 12 to capture quantitative measurements and utilizing a remote access device 40 in wireless communication with the fully enclosed flow chamber 12 to rapidly process host data through artificial intelligence processing units 42. To prevent coagulation and contamination of the aqueous biological material 44, the method employs a fully enclosed flow chamber 12 composed of biocompatible material 48.

The method further includes attaching memory cartridges 38 to the fully enclosed flow chamber 12 and removably connecting the clinical specimen collection equipment 46 at an injection point 24 of a passageway 20 located at the bottom surface 16. This arrangement facilitates the rapid transmission of the aqueous biological material 44 through a plurality of flow channels 28 fixedly attached within the interior space 18. By providing a passageway 20 as a substantially circular hollow tube 22 on a substantially vertical axis, the method enables a pump force insertion of the aqueous biological material 44 via a manual user-actuated plunger mechanism 66 attached to the clinical specimen collection equipment 46.

The application of the pump force moves the material rapidly through the passageway 20 into a central distribution hub 26, and subsequently through the flow channels 28 which are provided in an interconnected branched arrangement. The method involves directing the material to the outer ends 32 of the flow channels 28, where it is received by the plurality of microtubes 30. By positioning the plurality of microtubes 30 below the vertical plane 50 of the central distribution hub 26 and beneath the top surface 14, the method prevents flow reversal of the aqueous biological material 44 and ensures the material remains in a stationary position within the interior compartments 34 for individual analysis.

During the analysis phase, the method utilizes the medical diagnostic sensors 36 to track in vivo and in vitro physical and chemical parameters 68 of the host 62. This includes applying standard electrochemical principles—such as pH detection, ion-selective electrodes (Na, K, Ca), and neurotransmitter detection—once the aqueous biological material 44 is pumped into the flow channels 28. The artificial intelligence processing units 42 incorporate machine learning algorithms to analyze data in real time, exchanging information between the memory cartridges 38 and medical diagnostic sensors 36 via a local internal bus 58. This process employs predictive analytics to enhance measurement accuracy and executes artificial intelligence verification of the monitored data.

Finally, the method includes enclosing the radial arrangement of the flow channels 28, passageway 20, and microtubes 30 by fixing a glass slide 56 horizontally across the top surface 14. The remote access device 40 enables remote monitoring and control of the system, interpreting measured signals 70 to provide quantitative measurements of concentration, flow rate, and cellular characteristics. The method may perform a five-point analysis through the plurality of microtubes 30, allowing for simultaneous analysis of physical and chemical parameters 68 to provide a comprehensive diagnostic workup.

The method further provides for the integrated analysis system 10 to facilitate the collection of an aqueous biological material 44 by removably connecting the fully enclosed flow chamber 12 to clinical specimen collection equipment 46. In addition to a pre-filled syringe 64, the method may utilize various types of clinical specimen collection equipment 46 commonly used in healthcare settings, such as blood collection tubes, vacutainers, swabs, specimen containers, or needles, to ensure the proper collection, preservation, and transport of samples into the microtubes 30. To protect the aqueous biological material 44 and the radial arrangement of the flow channels 28, the method includes horizontally extending a glass slide 56 across the top surface 14 and fixing it thereon. As an alternative embodiment of the method, the user may select other cover materials for the fully enclosed flow chamber 12, such as quartz, plastic, or silicone coverslips, based on required optical properties, chemical resistance, and durability.

For data management and system oversight, the method utilizes at least one remote access device 40 to enable the remote monitoring and control of the integrated analysis system 10. The medical diagnostic sensor 36 integrated within the interior compartments 34 of the microtubes 30 is configured to measure one or more parameters 68 of the aqueous biological material 44, including but not limited to, concentration, flow rate, or cellular characteristics. By maintaining the medical diagnostic sensor 36 in direct contact with the aqueous biological material 44, the method accurately captures measured signals 70. These measured signals 70 may include scattered light measurements, fluorescence, or changes in electrical properties, which are then processed and interpreted by the remote access device 40 via the artificial intelligence processing units 42 to provide a quantitative measurement of the parameters 68 being assessed.

### Measuring, Recording, and Monitoring Data

The method of the integrated analysis system 10 as provided herein is designed to provide preliminary or detailed information that is reliable and can immediately guide clinical decisions in emergency situations. For example, in the case of a trauma patient where a blood count is immediately required, the method involves using the device to determine the need for a blood transfusion or the administration of additional blood or crystalloid fluids. When utilizing more than one microtube 30 to measure multiple parameters 68, the method includes the artificial intelligence processing units 42operating in cooperation with the history of the host 62 to provide early examination findings. The artificial intelligence processing units 42 facilitate locating a differential diagnosis, providing therapeutic recommendations, and managing test results from the medical diagnostic sensors 36. These monitoring steps can be performed on-scene, allowing a first-responder to initiate diagnostics and initial medical management using the available artificial intelligence and medical diagnostic sensor 36 information. Such recommendations may include supplying blood and fluids, improving breathing support, or providing ventilation based on cardiac, respiratory, or trauma-related etiologies.

In the case of an unconscious host 62, the method includes utilizing the system 10 to recommend a blood gas analysis to determine the necessary respiratory support or fluid volume. The method guides immediate resuscitation or fluid decisions by providing a blood gas analysis, including the partial pressure of carbon dioxide, partial pressure of oxygen, and base excess, to indicate the lung and metabolic status of the host 62. The method further provides for whole blood diagnostics, including blood type determination and evaluation, hematologic concerns such as a complete blood count, and biochemical analysis of electrolytes and liver function. Additionally, the method allows for the measurement of biomarkers including C-reactive protein, procalcitonin, and interferon via the medical diagnostic sensor 36 to provide a comprehensive quantitative measurement of the parameters 68 being assessed.

### Biological Materials

In various clinical settings, the method comprises collecting aqueous biological material 44, such as blood or spinal fluid, from a host 62 for diagnostic purposes. While the integrated analysis system 10 is frequently employed for human patients, the methodology further provides for diagnostic testing, disease monitoring, and research in veterinary settings, biomedical research on non-human primates, or the analysis of any organism that harbors pathogens or parasites. By utilizing the medical diagnostic sensor 36 to capture measured signals 70 across diverse biological environments, the method ensures that the artificial intelligence processing units 42 can provide reliable quantitative measurements regardless of the host 62 species.

The methodology involves managing the type and amount of aqueous biological material 44 placed within each microtube 30, which typically includes a fraction of a CC (e.g., 0.1-0.3 CC), though this volume may be reduced or increased based on the specific medical response scenario and setting. For example, the method includes detecting bacterial or viral pathogens within a small sample using integrated PCR technology as a medical diagnostic sensor 36. In such an embodiment, the medical diagnostic sensor 36 is prepared with a specific film to detect target viruses or bacteria to provide a bacterial match in a blood sample. Furthermore, the method provides for using smaller samples of aqueous biological material 44 for non-invasive prenatal DNA testing, wherein a mother's blood is analyzed to determine fetal risk for certain abnormalities by evaluating small fragments of cell-free DNA circulating in the maternal bloodstream.

### Hematological Analysis (Secondary Analysis)

As illustrated in FIG. 1, the method provides for secondary analysis following the preliminary or in-the-field analysis performed by the integrated analysis system 10. For example, the method may include further hematologic analysis at a core laboratory to determine a complete blood count, including white blood cells, hematocrit, hemoglobin, and platelet count. Following the complete labeling of the aqueous biological material 44 for further diagnosis and immunological analysis, the method provides for the differentiation of white blood cells using a flow cytometry process to automate the results. During this process, the artificial intelligence processing units 42 execute a learning protocol, improving the accuracy of new output data and results over time. By utilizing specific machine learning algorithms, the artificial intelligence processing units 42 process the stored data from the memory cartridge 38 to provide additional clinical conclusions and interpretations.

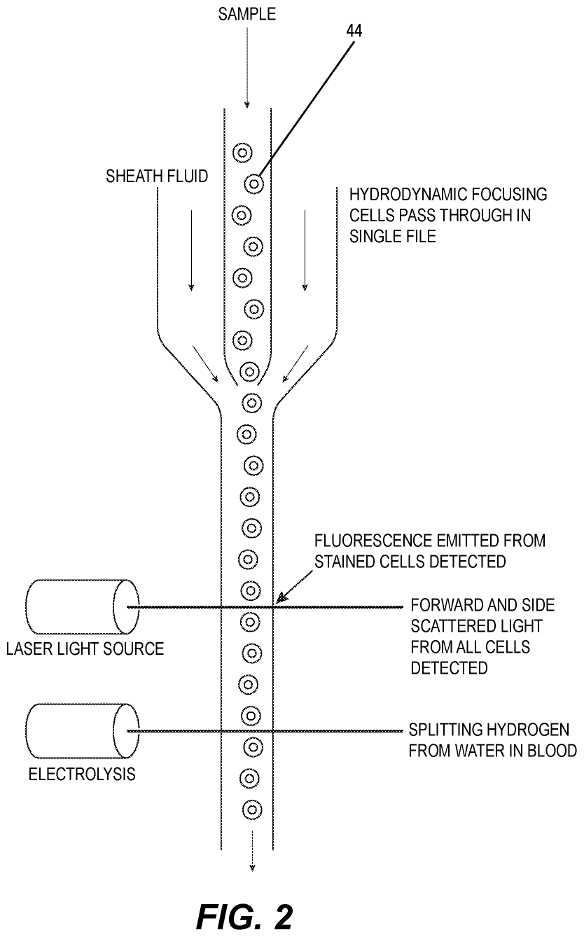

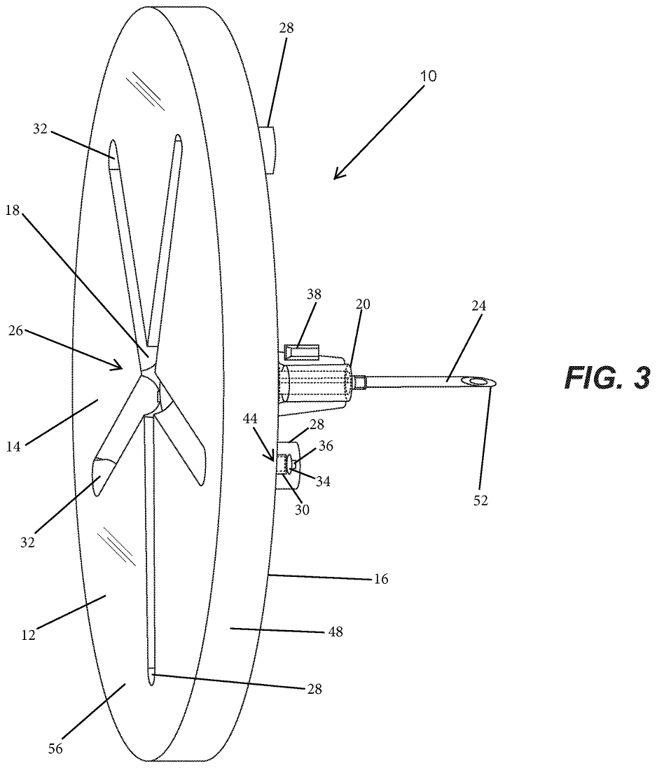

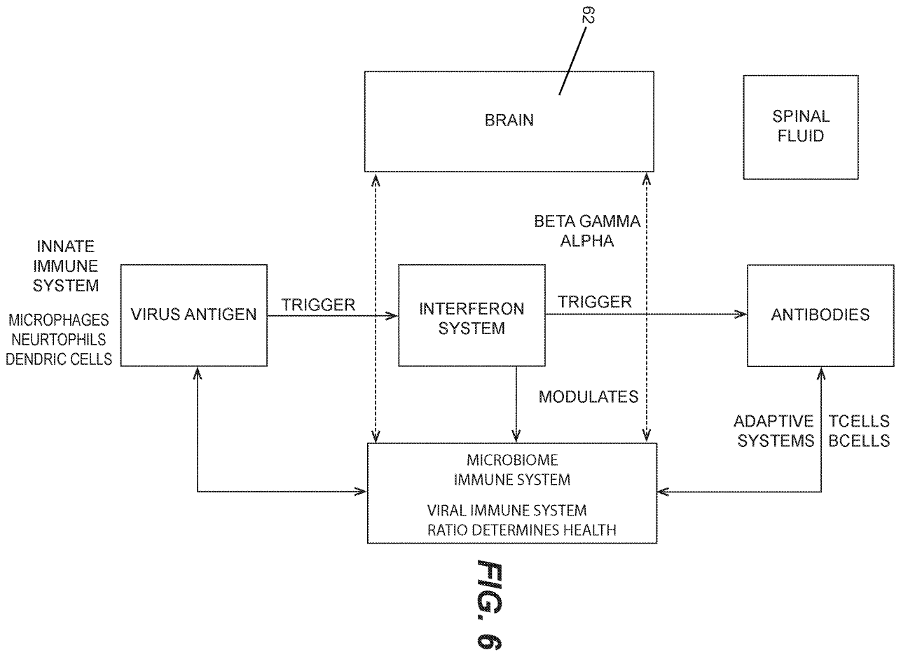

As shown in FIG. 2, the methodology further includes performing a secondary cellular analysis to detect the presence of specific immune cells and discern the distribution of immune cells within a sample. With reference to FIG. 3 and FIG. 6, the method involves utilizing specific algorithms within the artificial intelligence processing units 42 to identify the positive or negative influences of inflammation on the brain-intestine axis. The method tracks how an inflammatory response in the GI system may correlate with inflammation in the brain, such as by measuring increased levels of interferon released into the aqueous biological material 44. By utilizing the medical diagnostic sensor 36 to analyze spinal fluid, the method detects the possibility of infection or inflammatory processes affecting the brain or nerve fibers as a non-invasive alternative to a brain biopsy. The method further involves monitoring mediators and pro-inflammatory or anti-inflammatory proteins released by immune cells to assess their impact on both local environments, such as the bowels, and remote organs. Through the analysis of parameters 68 within the microbiome, the method enables the identification of contributing factors to neuro-developmental problems, such as inflammatory responses causing tissue scarring or affecting synapse development, rather than relying solely on the detection of a brain bleed.

### Flow Chamber

Overall, the design of the fully enclosed flow chamber 12 and the microtube 30 used to hold a medical diagnostic sensor 36 and aqueous biological material 44 is aimed at providing a convenient and reliable repository for the measurement of specific parameters in such materials, while ensuring safety, reliability, accuracy, and ease of use in a clinical or point-of-care setting.

The fully enclosed flow chamber 12 and associated parts can be constructed in various sizes and can be provided as standard construction including the provision of durable materials for all elements as provided herein. It can be produced in a circular shape, a standard box-like shape or provided as desired by a particular healthcare facility. In a healthcare setting, the shape of a medical device plays a crucial role in facilitating easy and efficient handling of biological materials such as blood. The device's ergonomic design ensures that it can be comfortably held, operated, and easily integrated into the workflow of a healthcare professional.

The design of the fully enclosed flow chamber 12 and microtubes 30 can vary depending on the specific application and the type of medical diagnostic sensors 36 being used and can be made of biocompatible materials 48 to ensure that the materials do not alter the composition of the biological materials. Common materials might include medical-grade plastics or polymers. The biocompatible material 48 can be chosen to minimize interaction with whole blood or similar biological materials, thereby preserving the original sample compositions and prevent coagulation and similar conditions that can affect the analysis. The fully enclosed flow chamber 12 and microtubes 30 can be designed to be disposable and single-use to prevent cross-contamination and ensure the accuracy of subsequent measurements.

The microtube 30 may include features such as capillary action channels or microfluidic pathways to enable the efficient movement of the aqueous biological material 44 to the medical diagnostic sensor 36. The microtube 30 includes interior compartments 34 where the medical diagnostic sensor 36 is inserted and the medical diagnostic sensor 36 can be positioned to have direct contact with the biological materials and thereby allowing for the measurement of the desired parameter. The design of the microtube 30 ensures that the medical diagnostic sensor 36 remains securely in place and properly aligned to enable accurate measurements. In some cases, the microtube 30 may include electrical contacts, optical windows, or other local internal bus 58 interfaces that allow the medical diagnostic sensor 36 to communicate with external measurement or analysis equipment. The microtube 30 may also include features to ensure proper alignment and connection with the measurement device or instrument.

The protective cover is a safety feature that prevents accidental exposure to the biological material. Additional safety features such as tamper-evident seals for the fully enclosed flow chamber 12 and the medical diagnostic sensor 36 compartment may also be incorporated to minimize the risk of exposure to biohazards during the handling and disposal of the microtube 30. The fully enclosed flow chamber 12 and the microtube 30 may also include markings, labels, or identifiers to convey important information such as the type of medical diagnostic sensor 36, lot number, expiration date, and any specific handling instructions.

### Artificial Intelligence (AI)

The method provides for the integration of artificial intelligence processing units 42 based on the specific requirements and constraints of a facility, utilizing a local internal bus 58 to facilitate the exchange of data between the memory cartridges 38, medical diagnostic sensors 36, and the artificial intelligence processing units 42. This integration may involve establishing wireless connections via a wireless transmitter 60 or utilizing wired connections to incorporate artificial intelligence into the evaluation system. The method leverages this architecture to enhance the accuracy and efficiency of the analysis of the aqueous biological material 44 and to improve the effectiveness of medical diagnostics and patient care. Specifically, the method includes identifying patterns, anomalies, and trends within the biological sample data via the artificial intelligence processing units 42 to assist in diagnosing medical conditions, predicting patient outcomes, and providing insights for medical research. Furthermore, the methodology involves processing data to reinforce the medical conclusions of healthcare professionals by providing data-driven insights and recommendations to support the construction of clinical opinions regarding specific treatment methods or plans, while ensuring adherence to regulatory standards, privacy considerations, and ethical guidelines.

### Memory Cartridges

The method further comprises configuring the mechanical, wireless, and electrical connections of the integrated analysis system 10 according to the specific design requirements of the memory cartridge 38, the medical diagnostic sensors 36, and the specific evaluation of the aqueous biological material 44. In a medical context, the methodology includes adhering to relevant industry and regulatory standards for medical devices to ensure the safety and effectiveness of all system interconnections. By managing the interface between the memory cartridge 38 and the medical diagnostic sensor 36, the method ensures the reliable capture and storage of parameters 68 and measured signals 70 during the biological material evaluation process.

Certain components necessary to the operation of the memory device or other parts of the fully enclosed flow chamber 12 are not shown or described in detail because they are components well known to those in the relevant arts. These components include well-known components, attachments, parts, and operations. For example, a memory cartridge 38 in a general sense is a storage device designed to hold digital data and the function of the memory cartridge 38 will vary depending on the specific application in a healthcare setting. A few common functions of the memory cartridge 38 as provided herein can include storage and retrieval of digital data such as files, documents, images, and software.

Physically connecting memory cartridges 38 to a fully enclosed flow chamber 12 and to a series of medical diagnostic sensors 36 for evaluating samples of aqueous biological material 44 involves a plurality of considerations regarding mechanical, wireless, and electrical connections. The design as provided herein considers the compatible physical dimensions and connections between a specific memory cartridge 38 and a fully enclosed flow chamber 12 to ensure a secure mechanical connection. Given the medical nature of the fully enclosed flow chamber 12, the mechanical connection should be durable and able to withstand repeated use and potential sterilization processes. The mechanical connection should be designed for easy insertion and removal to facilitate the workflow of biological material evaluation.

If wireless communication is used, the memory cartridge 38 and medical diagnostic sensors 36 should support a reliable wireless protocol for transmitting data via a wireless transmitter 60 to the remote access device 40. This could be Bluetooth, Wi-Fi, or another suitable wireless standard. If sensitive patient data is being transmitted wirelessly, the connection should incorporate appropriate security measures to protect the data from unauthorized access or interception.

The fully enclosed flow chamber 12, memory cartridge 38, and medical diagnostic sensors 36 can have a consistent power supply, whether through batteries, charging, or another power source and the electrical connection can support the transfer of data via the local internal bus 58 between the memory cartridge 38, the medical diagnostic sensors 36, and ensuring that information from the biological material evaluation is accurately stored and accessible.

The method provides for the physical connecting of a memory cartridge 38 to a fully enclosed flow chamber 12 and to a series of medical diagnostic sensors 36 to evaluate samples of aqueous biological material 44. This process involves managing compatible physical dimensions and connections between a specific memory cartridge 38 and the fully enclosed flow chamber 12 to ensure a secure mechanical connection. The methodology includes utilizing mechanical connections designed for durability to withstand repeated use and potential sterilization processes, while facilitating the workflow of biological material evaluation through easy insertion and removal.

If wireless communication is employed, the method involves utilizing a memory cartridge 38 and medical diagnostic sensors 36 that support a reliable wireless protocol for transmitting data via a wireless transmitter 60 to the remote access device 40. This step may include implementing protocols such as Bluetooth, Wi-Fi, or other suitable wireless standards. Furthermore, when transmitting sensitive patient data, the method includes incorporating appropriate security measures to protect the data from unauthorized access or interception during wireless transmission.

The methodology further comprises maintaining a consistent power supply for the fully enclosed flow chamber 12, memory cartridge 38, and medical diagnostic sensors 36, whether through batteries, charging, or an alternative power source. This ensures that the electrical connections support the transfer of data via the local internal bus 58 between the memory cartridge 38 and the medical diagnostic sensors 36. By maintaining these power and data interfaces 72, the method ensures that information from the biological material evaluation, including all parameters 68 and measured signals 70, is accurately stored and remains accessible for analysis.

While the invention has been described in detail and with reference to specific embodiments thereof, it will be apparent to one skilled in the art that various changes and modifications can be made therein without departing from the spirit and scope thereof.

What is claimed is:

1. A method for integrated blood analysis using an integrated analysis system, the method comprising:
providing a fully enclosed flow chamber having a top surface, a bottom surface, and an interior space defined between the top and bottom surfaces;
introducing an aqueous biological material through a passageway extending along a substantially vertical axis through the flow chamber, the passageway comprising a substantially circular hollow tube defining an injection point at the bottom surface;
distributing the biological material through a central distribution hub located within the interior space and fluidly connected to the passageway;
directing the biological material through a plurality of flow channels monolithically integrated within the flow chamber and extending radially from the central distribution hub;
receiving the biological material into a plurality of microtubes located at outer ends of the flow channels, the microtubes comprising interior compartments;
analyzing the biological material via at least one medical diagnostic sensor embedded within the interior compartments of the microtubes;
storing data on at least one memory cartridge attached to the fully enclosed flow chamber; and
processing data via a remote access device comprising artificial intelligence (AI) processing units in wireless communication with the memory cartridge and the medical diagnostic sensor;
wherein the injection point receives the aqueous biological material from a clinical specimen collection equipment removably attached thereto, such that a pump force moves the biological material from the injection point, through the central distribution hub, and into the plurality of microtubes for stationary sensor analysis.

2. The method of claim 1, wherein the fully enclosed flow chamber is composed of a biocompatible material.

3. The method of claim 1, wherein the plurality of microtubes are positioned below a vertical plane of the central distribution hub and beneath the top surface of the flow chamber, such that the structural arrangement prevents flow reversal of the aqueous biological material.

4. The method of claim 1, further comprising forming a hermetic seal at the injection point of the passageway via a female Luer lock connector configured to receive a corresponding male Luer connector of the clinical specimen collection equipment.

5. The method of claim 1, further comprising sealing the radial arrangement of the flow channels and microtubes via a glass slide horizontally extending across the top surface of the flow chamber and fixed thereon.

6. The method of claim 1, further comprising performing quantitative measurements of the biological material via an optical sensor without prior analyte concentration or separation.

7. The method of claim 1, further comprising measuring at least one physical parameter and at least one chemical parameter of the aqueous biological material simultaneously using electrochemical principles via the medical diagnostic sensor.

8. The method of claim 1, further comprising transferring data between the memory cartridge and the medical diagnostic sensor via a local internal bus within the sealed flow chamber.

9. The method of claim 1, further comprising executing predictive analytics via the AI processing unit to analyze biometric data in real time and transmitting final diagnostic results, rather than raw sensor data, via a wireless transmitter.

10. The method of claim 1, further comprising performing individual preliminary analysis of the aqueous biological material across at least five distinct analysis points via the plurality of microtubes.

11. The method of claim 1, further comprising connecting at least one of the medical diagnostic sensors to a host to track in vivo and in vitro physical parameters.

12. The method of claim 1, further comprising providing the pump force insertion via a pre-filled syringe containing the aqueous biological material, the syringe comprising a manual user-actuated plunger mechanism coupled to the injection point.

13. The method of any one of claims 1, 2, or 3, further comprising distributing the material simultaneously into the plurality of flow channels via the central distribution hub located within the interior space in fluid communication with the vertical passageway.

14. The method of any one of claims 1, 2, or 3, further comprising coupling an outlet of a pre-filled syringe containing the aqueous biological material to the injection point of the passageway, the syringe comprising a barrel and a plunger.

15. The method of any one of claims 1, 2, or 3, further comprising performing quantitative measurements on the material within the flow channel via a plurality of optical sensors without prior analyte concentration or separation.
